(19) United States
(12) Patent Application Publication
(10) Pub. No.: US 2019/0247525 A1
(43) Pub. Date: Aug. 15, 2019

(54) Copper Sulfide Perfluorocarbon Nanocarriers

(71) Applicant: Georgia Tech Research Corporation, Atlanta, GA (US)

(72) Inventors: Daniela Yvonne Santiesteban, Spring, TX (US); Diego S. Dumani, Atlanta, GA (US); Stanislav Emelianov, Atlanta, GA (US)

(21) Appl. No.: 16/274,982

(22) Filed: Feb. 13, 2019

Related U.S. Application Data
(60) Provisional application No. 62/629,969, filed on Feb. 13, 2018.

(51) Int. Cl. A61K 49/22; A61K 9/51; A61K 9/50; A61K 47/69; A61K 49/18; A61K 41/00; G06T 7/00

(52) U.S. Cl. CPC A61K 49/222; A61K 9/5115; A61K 9/5123; A61K 9/5015; G06T 2207/10132; A61K 49/1824; A61K 41/0028; A61K 9/5192; G06T 7/0012; A61K 47/6929

(57) ABSTRACT

The present invention provides nanocarrier compositions, for example, copper sulfide perfluorocarbon nanocarrier compositions, and methods of making the same. The compositions are useful for imaging, diagnostics, therapy and for other uses.

This application claims the benefit of priority of our prior U.S. provisional application Ser. No. 62/629,969, filed Feb. 13, 2018, the disclosure of which is hereby incorporated by reference in its entirety.

### FIELD OF THE INVENTION

The present invention provides nanocarrier compositions, for example, copper sulfide perfluorocarbon nanocarrier compositions, and methods of making the same. The compositions are useful for imaging, diagnostics, therapy and for other uses.

### BACKGROUND OF THE INVENTION

Nanotechnology-based tools for biomedical applications are widely sought. However, progress in the use of nanoparticles in clinical and diagnostic purposes has remained limited (Kiessling, F. et al., Nanoparticles for imaging: Top or flop? Radiology, 2014, 273 (1), 10-28). Nanoparticles can be classified into two groups: 1) organic (i.e. liposomes, micelles, polymeric complexes, etc.) and inorganic (i.e. metallic, semiconductor, silica, etc); each group having inherent strengths and weaknesses. Organic nanoparticles are valuable therapeutic carriers and are biodegradable and effectively cleared. However, they make poor diagnostic agents because they typically fail to provide image contrast on their own. On the other hand, most inorganic nanoparticles are strong contrast agents but have limited therapeutic carrier capacity and poor biodegradability and clearance (Anselmo, A. C. et al., A review of clinical translation of inorganic nanoparticles. The AAPS journal, 2015, 17 (5), 1041-1054). Thus, many research groups have now turned to combination nanoparticle products (Cheon, J. et al., Synergistically integrated nanoparticles as multimodal probes for nanobiotechnology. Accounts of chemical research, 2008, 41 (12), 1630-1640; Huynh, E. et al., Multimodal micro, nano, and size conversion ultrasound agents for imaging and therapy. Wiley Interdisciplinary Reviews: Nanomedicine and Nanobiotechnology 2016). However, although combination nanoparticles may augment the strengths of individual particles, they are unable to overcome inherent weaknesses. For instance, combinations can result in increased contrast, but at the expense of biocompatibility or clearance limitations.

Perfluorocarbon nanodroplets are being studied for various applications but are most commonly used in ultrasound imaging (Wilson, K. et al., Biomedical photoacoustics beyond thermal expansion using triggered nanodroplet vaporization for contrast-enhanced imaging. Nature communications 2012, 3, 618; Sheeran, P. S. et al, Formulation and acoustic studies of a new phase-shift agent for diagnostic and therapeutic ultrasound. Langmuir, 2011, 27 (17), 10412-10420; Kripfgans, O. D. et al., Acoustic droplet vaporization for therapeutic and diagnostic applications. Ultrasound in medicine & biology 2000, 26 (7), 1177-1189; Partlow, K. C. et al., 19F magnetic resonance imaging for stem/progenitor cell tracking with multiple unique perfluorocarbon nanobeacons. The FASEB Journal 2007, 21 (8), 1647-1654). Ultrasound is emerging as one of the most widely used diagnostic imaging modalities due to its low cost, portability, and ability to safely provide real-time structural information with clinically relevant spatial resolutions (Klibanov, A. L., Targeted delivery of gas-filled microspheres, contrast agents for ultrasound imaging. Advanced drug delivery reviews 1999, 37 (1), 139-157; Foster, F. S. et al., Ultrasound for the visualization and quantification of tumor microcirculation. Cancer and Metastasis reviews 2000, 19 (1-2), 131-138). Perfluorocarbon nanodroplets represent the submicron, liquid version of the ubiquitously used ultrasound contrast agent, perfluorocarbon microbubbles. Compared to microbubbles, perfluorocarbon nanodroplets offer several advantages including increased circulation lifetimes and the ability to extravasate hyperpermeable vasculature and directly diagnose and treat diseased tissue (Rapoport, N., Drug-Loaded Perfluorocarbon Nanodroplets for Ultrasound-Mediated Drug Delivery. In Therapeutic Ultrasound, Springer: 2016; pp 221-241). Furthermore, perfluorocarbon nanodroplets can be phase-changed into microbubbles, via acoustic or laser irradiation and maintain the same ultrasound enhancing properties. Of the two perfluorocarbon subsets, those that are laser-triggered are advantageous for several reasons (Hannah, A. et al., Indocyanine green-loaded photoacoustic nanodroplets: dual contrast nanoconstructs for enhanced photoacoustic and ultrasound imaging. ACS nano 2013, 8 (1), 250-259; Giesecke, T. et al., Ultrasound-mediated cavitation thresholds of liquid perfluorocarbon droplets in vitro. Ultrasound in medicine & biology 2003, 29 (9), 1359-1365). Laser-triggered perfluorocarbon nanodroplets are safer, the activation event is better controlled, and they are more comprehensive imaging agents because they supplement ultrasound contrast with photoacoustic contrast. Photoacoustic imaging is a novel imaging technique that works synergistically with ultrasound and provides molecularly specific contrast at the spatial resolution of ultrasound (Mallidi, S. et al., Photoacoustic imaging in cancer detection, diagnosis, and treatment guidance. Trends in biotechnology 2011, 29 (5), 213-221). The Perfluorocarbon nanodroplet vaporization event emits an intense photoacoustic signal, which contains valuable information that would be lost to interference with the ambient ultrasound field if perfluorocarbon nanodroplets were acoustically triggered. Additionally, laser-triggered perfluorocarbon nanodroplets require the incorporation of a photoabsorber to initiate the phase-change, which has the potential for long-term photoacoustic contrast and therapeutic applications. Because of the crucial role photo-absorbers play in laser-activated perfluorocarbon nanodroplets, choosing the right photo-absorber is essential in attaining clinical relevance and future translation.

Laser-activated perfluorocarbon nanodroplets are made with various photo-absorbers ranging from organic dyes to inorganic nanoparticles (Wilson, K. et al., Biomedical photoacoustics beyond thermal expansion using triggered nanodroplet vaporization for contrast-enhanced imaging. Nature communications 2012, 3, 618; Hannah, A. et al., Indocyanine green-loaded photoacoustic nanodroplets: dual contrast nanoconstructs for enhanced photoacoustic and ultrasound imaging. ACS nano 2013, 8 (1), 250-259; Wei, C. et al., Nonlinear contrast enhancement in photoacoustic molecular imaging with gold nanosphere encapsulated nanoemulsions. Applied physics letters 2014, 104 (3), 033701; Paproski, R. J. et al., Porphyrin Nanodroplets: Sub-micrometer Ultrasound and Photoacoustic Contrast Imaging Agents. Small 2016, 12 (3), 371-380; Hannah, A. S. et al., Photoacoustic and ultrasound imaging using dual contrast perfluorocarbon nanodroplets triggered by laser pulses at 1064 nm. Biomedical optics express 2014, 5 (9), 3042-3052). The ideal photo-absorber absorbs at long wavelengths for increased penetration resulting in a higher signal to noise ratio, has good photothermal stability and efficiency, and is biodegradable with good clearance properties. Thus far, photo-absorbers used as perfluorocarbon nanodroplet triggers have struggled to fulfill these requirements. For instance, gold nanoparticles can be synthesized to absorb within desirable wavelengths (˜1064 nm); however, they are not easily biodegradable and cannot be cleared in a timely manner, provoking concerns over long-term biocompatibility (Hannah, A. S. et al., Photoacoustic and ultrasound imaging using dual contrast perfluorocarbon nanodroplets triggered by laser pulses at 1064 nm. Biomedical optics express 2014, 5 (9), 3042-3052). Also, upon pulsed laser irradiation they are prone to melting and morphing shape, altering their spectrum and thus limiting long-term applications (Chen, Y. S. et al., Enhanced thermal stability of silica-coated gold nanorods for photoacoustic imaging and image-guided therapy. Optics express 2010, 18 (9), 8867-8878). Many dyes have been utilized as triggers, but the majority absorb in the NIR or at shorter wavelengths, resulting in reduced penetration depths. In addition, dyes are susceptible to photobleaching, once again limiting long-term PA application (Hannah, A. et al., Indocyanine green-loaded photoacoustic nanodroplets: dual contrast nanoconstructs for enhanced photoacoustic and ultrasound imaging. ACS nano 2013, 8 (1), 250-259; Paproski, R. et al., Porphyrin Nanodroplets: Sub-micrometer Ultrasound and Photoacoustic Contrast Imaging Agents. Small 2016, 12 (3), 371-380). Engineering laser-activated perfluorocarbon nanodroplets with better photoabsorber characteristics is crucial in developing effective and clinically relevant imaging and therapeutic agents.

Copper sulfide nanoparticles are inorganic, semi-conductor nanoparticles primarily used as photothermal agents due to their photothermal stability (Zhou, M. et al., A chelator-free multifunctional [64Cu] CuS nanoparticle platform for simultaneous micro-PET/CT imaging and photothermal ablation therapy. Journal of the American Chemical Society 2010, 132 (43), 15351-15358; Hessel, C. M. et al., Copper selenide nanocrystals for photothermal therapy. Nano letters 2011, 11 (6), 2560-2566; Li, Y. et al., Copper sulfide nanoparticles for photothermal ablation of tumor cells. Nanomedicine 2010, 5 (8), 1161-1171). Copper sulfide nanoparticles are non-cytotoxic, biodegradable, and are cleared within a reasonable timeframe (Guo, L. et al., A comparative study of hollow copper sulfide nanoparticles and hollow gold nanospheres on degradability and toxicity. ACS nano 2013, 7 (10), 8780-8793). They absorb within the second optical imaging window (1000 nm to 1350 nm), ideal for achieving clinically relevant penetration depths with increased signal to noise ratio (Smith, A. M. et al., Bioimaging: Second window for in vivo imaging. Nat Nano 2009, 4 (11), 710-711). One drawback of copper sulfide nanoparticles is their lower photothermal efficiency compared to gold nanoparticles, which necessitates significant copper sulfide nanoparticle concentrations for enhanced photoacoustic contrast (Goel, S. et al., Synthesis and biomedical applications of copper sulfide nanoparticles: from sensors to theranostics. Small 2014, 10 (4), 631-645).

Thus, new or improved imaging and therapeutic agents are continually needed that have low toxicity, high imaging capacity and other advantageous features. The compositions and methods described herein are directed to these and other ends.

### SUMMARY OF THE INVENTION

The present invention provides nanocarrier compositions and methods of using them in imaging and therapy. The nanocarrier compositions synergistically combine organic and inorganic components for enhanced image contrast with high potential for clinical translation. The nanocarriers are biocompatible, can be imaged at clinically relevant depths, provide enhanced ultrasound and photoacoustic contrast, and have the capability of delivering therapeutics, as well as other advantageous features. For example, applicants have discovered that the prior art limitations can be overcome when combining, e.g., copper sulfide nanoparticles with laser-activated perfluorocarbon nanodroplets since the nanodroplet vaporization gives intense photoacoustic signal and ultrasound contrast, with less amounts of copper sulfide nanoparticles required. Hence, combining copper sulfide nanoparticles with perfluorocarbon nanodroplets synergistically overcomes the individual limitations when copper sulfide nanoparticles and/or the laser-activated perfluorocarbon nanoparticles are used on their own.

In certain embodiments, the present invention provides a nanocarrier comprising: a perfluorocarbon solution comprising a plurality of nanoparticles dispersed therein (and/or on the surface); and a coating material disposed around the exterior surface of the perfluorocarbon solution. In some embodiments, the nanoparticles comprise fluorinated copper sulfide nanoparticles and the coating material is a fluorosurfactant.

The invention also provides a method of preparing a nanocarrier composition comprising: providing a nanoparticle precursor, fluorinating the nanoparticle precursor, and contacting the fluorinated nanoparticle with a perfluorocarbon solution and a coating material to afford a nanocarrier agent.

The present invention further provides a method of imaging comprising: contacting a biological tissue with a nanocarrier agent comprising a perfluorocarbon solution comprising a plurality of nanoparticles dispersed therein, and a coating material disposed around the exterior surface of the perfluorocarbon solution; applying energy to the tissue, wherein the applying energy results in at least partial vaporization of the nanocarrier agent; and imaging the biological tissue.

The invention additionally provides a method of therapy comprising: contacting a biological tissue with a nanocarrier agent comprising a perfluorocarbon solution comprising a plurality of nanoparticles dispersed therein, and a coating material disposed around the exterior surface of the perfluorocarbon solution; and applying energy to the tissue, wherein the applying energy results in at least partial vaporization of the nanocarrier agent.

The features and advantages of the present invention will be apparent to those skilled in the art. While numerous changes may be made by those skilled in the art, such changes are within the spirit of the invention.

### BRIEF DESCRIPTION OF THE DRAWINGS

FIG. 1 is an example illustration of a nanocarrier of the invention.

FIG. 2 shows the characterization of copper sulfide nanoparticles. (a) Absorbance spectrum of PEGylated and fluorinated copper sulfide nanoparticles. (b) Zeta potential measurements for PEGylated and fluorinated copper sulfide nanoparticles. (c) Transmission electron microscopy (TEM) image of fluorinated copper sulfide nanoparticles. Scale bar, 50 nm. (d) Image of fluorinated copper sulfide nanoparticles (left) and PEGylated copper sulfide nanoparticles (right) in perfluorocarbon solution. (e) Results of dynamic light scattering experiment measuring the hydrodynamic diameter of nanocarrier agents.

FIG. 3 shows the results of an experiment measuring the absorbance of gold (a) and copper sulfide (b) nanoparticles at 0 to 100 mJ/cm².

FIG. 4 shows the results of a phantom imaging experiment. (a) Schematic of phantom setup; imaging plane is represented by dashed square. (b) Ultrasound and photoacoustic frames show contrast data retrieved for nanocarrier composition at 0, 2 s, and 20 s intervals. (c) Normalized ultrasound and photoacoustic plots for data collected in (b).

FIG. 5 shows the results of a mouse imaging experiment. (a) 3-D doppler ultrasound image of enlarged mouse lymph node and vasculature. (b) Schematic of mouse experiment. (c) Ultrasound and photoacoustic contrast images of lymph node, at pre-injection, start of irradiation, and post-irradiation time points. (d) Ultrasound and photoacoustic contrast plots for data collected during irradiation period.

FIG. 6 shows the results of a mouse imaging experiment. (a) Ultrasound contrast inside the lymph node for mouse treated with nanocarrier composition. (b) Image of lymph node and as surrounding vasculature for mouse treated with nanocarrier composition.

FIG. 7 shows the results of a mouse imaging experiment. (a) Ultrasound/photoacoustic image of spleen of mouse treated with nanocarrier composition. (b) Plot of photoacoustic signal of spleen and skin of mouse treated with nanocarrier composition.

FIG. 8 shows the results of an imaging experiment. (a) Unprocessed photoacoustic signal at several time points during the irradiation sequence. (b) Temporal photoacoustic signals of pixels with perfluorocarbon nanocarrier and endogenous signal. Linear regression is performed on frames that capture decay. Slopes (−4.8 vs −0.38) with rates above a certain threshold are identified. (c) The processed image depicts location of perfluorocarbon nanodroplets (i.e., nanocarriers). Scale bar is 2 mm.

FIG. 9 shows the results of an imaging experiment. (a) Ultrasound signal at several different time points during the irradiation sequence. (b) Temporal ultrasound signals of pixels with perfluorocarbon nanodroplets and endogenous signal. Linear regression is performed on frames that capture decay. Slopes (2.47 vs 0.28) with rates above a certain threshold are identified. (c) The processed image depicts location of perfluorocarbon nanodroplets (i.e., nanocarriers). Scale bar is 2 mm.

### DETAILED DESCRIPTION

The present invention provides nanoparticle-containing carrier agents having several advantageous features, such as biocompatibility, biodegradability and the ability to provide medical imaging contrast at clinically relevant depths. These properties make the nanocarriers useful for a variety of applications, including drug delivery, diagnostics, therapy and imaging. In some embodiments, the size of the nanocarriers of the inventions allow for passive diffusion into biological tissues (such as tumor tissues) which allows for facile imaging of several pathologies such as cancer and inflammatory diseases and disorders. The size of the nanocarriers allow them to access most biological tissues in which imaging and/or therapy is needed. Carrier agents of the invention comprising metal nanoparticles and therapeutic agents function as optically triggered drug delivery and release systems.

In certain embodiments, the nanocarriers exhibit a capacity to be “remotely triggered.” In these embodiments, the nanocarrier system remains inert in the body until they are specifically triggered, e.g., by irradiating at least a portion of biological tissue with a light source or a radio frequency field. Nanocarriers may also be used advantageously in therapeutic applications by targeting the nanocarriers to a specified location, and then remotely triggering the nanocarriers to an activated state. This feature may minimize the side effects of systemic drugs, microwave ablation therapy, vessel occlusion therapy, photothermal therapy, and nuclear medicine. Nanocarriers of the invention may also be used as contrast agents for optical imaging methods (such as optical coherence tomography), magnetic resonance imaging, computed tomography, and photoacoustic imaging (for example, through mechanisms of vaporization and thermal expansion). Additionally, embodiments containing magnetic iron oxide and/or cobalt nanoparticles may provide nanocarriers that can be used in microwave ablation therapy and magnetomotive imaging enhancement.

### A. Nanocarrier Compositions

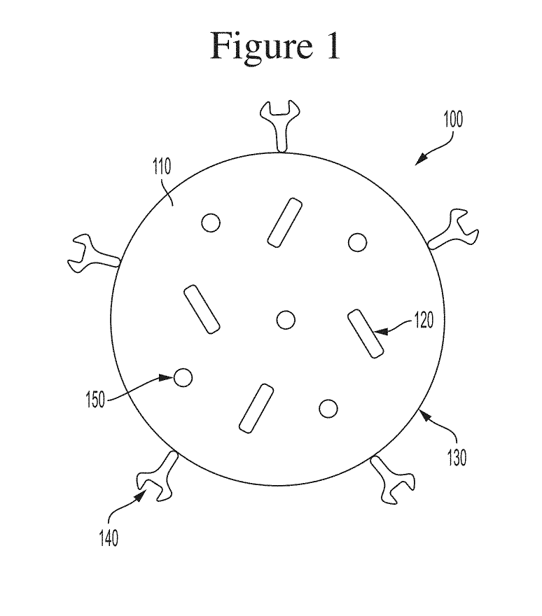

The nanocarriers of the invention may comprise an organic liquid (such as a perfluorocarbon solution) comprising a plurality of nanoparticles dispersed therein, and a coating material disposed around the exterior surface of the organic liquid. One exemplary embodiment of a nanocarrier of the present invention is illustrated in FIG. 1. In this embodiment, a nanocarrier 100 may comprise an organic liquid 110 and nanoparticles 120 dispersed throughout the organic liquid 110. Additionally, a nanocarrier of the present disclosure may further comprises a coating 130, which surrounds the exterior surface of organic liquid 110. The terms “coat,” “coated,” or “coating,” as used herein, refer to at least a partial coating of the organic liquid. One hundred percent coverage is not necessarily implied by these terms. Optionally, in some embodiments, a nanocarrier of the present invention may also comprise a targeting moiety 140 and/or a therapeutic agent 150.

Examples of organic liquids suitable for use in the nanocarriers of the present disclosure may include, but are not limited to, perfluorocarbons. The term “perfluorocarbon,” as used herein, includes compounds that contain only carbon and fluorine atoms, for example, saturated, unsaturated, and cyclic perfluorocarbons such as perfluoroalkanes such as perfluoromethane, perfluoroethane, perfluoropropanes, perfluorobutanes (e.g. perfluoro-n-butane, optionally in admixture with other isomers such as perfluoro-isobutane), perfluoropentanes, perfluorohexanes and perfluoroheptanes; perfluoroalkenes such as perfluoropropene, perfluorobutenes (e.g. perfluorobut-2ene) and perfluorobutadiene; perfluoroalkynes such as perfluorobut-2-yne, and perfluorocycloalkanes such as perfluorocyclobutane, perfluoromethylcyclobutane, perfluorodimethylcyclobutanes, perfluorotrimethylcyclobutanes, perfluorocyclopentane, perfluoromethycylopentane, perfluorodimethylcyclopentanes, perfluorocyclohexane, perfluoromethylcyclohexane and perfluorocycloheptane.). The saturated perfluorocarbons, have the formula CₙFₙ₊₂, where n is from 1 to 12, or from 2 to 10, or from 3 to 8 or from 3 to 6. Suitable perfluorocarbons include, for example, CF₄, C₂F₆, C₃F₈, C₄F₈, C₄F₁₀, C₅F₁₂, C₆F₁₂, C₇F₁₄, C₈F₁₈, and C₉F₂₀. In some embodiments, the perfluorocarbon is perfluoropentane, perfluorohexane, perfluorobutane, or perfluoroheptane, or a combination thereof.

The nanocarrier of the present invention can further comprise nanoparticles dispersed in the organic liquid. The term “nanoparticle” refers to a particle having a dimension in the range of about 1 nm to about 1 micron. For example, nanoparticles suitable for use in the nanocarrier compositions of the present invention may have an exterior diameter on the order of about 1 nanometers to about 1 micron, while in some embodiments, the diameter may range from about 100 nanometers to about 500 nanometers, or about 0.5 nanometers to about 50 nanometers. The nanoparticles may be any shape, including but not limited to, spheres, rods, shells, plates, crescents, and the like. Furthermore, in some embodiments, the nanoparticles also may have tunable properties so as to resonate in the NIR region. For example, by varying the shape and aspect ratio of nanoparticles, the particles can be manufactured to absorb light at the desired wavelength across a wide spectrum including near infrared spectrum.

Nanoparticles suitable for use in the present invention may comprise any biocompatible material, such as a biocompatible metal. Examples of suitable metals include, but are not limited to, copper, iron oxide, platinum, cobalt and noble metals, such as gold and/or silver. One specific example of a suitable metal is copper sulfide. In another embodiment, the nanoparticles comprise fluorinated copper sulfide. One of ordinary skill in the art will be able to select a suitable type of nanoparticle taking into consideration at least the type of imaging and/or therapy to be performed.

In some embodiments, nanoparticles may be included in the organic liquid in an amount less than about 1 milligram per milliliter. In some embodiments, nanoparticles may be included in the organic liquid in an amount less than about 10 micrograms per milliliter. In some embodiments, the nanoparticles may be included in the organic liquid in an amount of about 1.0 milligram per milliliter, or about 0.8 milligrams per milliliter, or about 0.7 milligrams per milliliter or about 0.5 milligrams per milliliter, or about 0.3 milligram per milliliter, or about 0.1 milligram per milliliter, or about 0.05 milligram per milliliter, or about 0.01 milligram per milliliter. In some embodiments, the nanocarrier is present in the organic liquid at a concentration of about 0.05 mg/mL to about 0.3 mg/mL. When used at this concentration, there are generally no cytotoxic effects due to the nanoparticles.

In addition to the organic liquid, a nanocarrier of the present disclosure further comprises a coating material (or stabilizing shell) disposed around the exterior surface of the organic liquid. Examples of suitable coating materials may include, but are not limited to, surfactants, lipids and proteins. Exemplary surfactants for use in the present invention include nonionic, anionic, cationic, amphoteric and zwitterionic surfactants. In some embodiments, the surfactant is a fluorosurfactant (for example, a nonionic ethoxylated fluorosurfactant). One specific example of a suitable fluorosurfactant is Zonyl® FSO fluorosurfactant. In some embodiments, the coating material is a lipid such as a phosphocholine derivative (e.g., 1,2-distearoyl-sn-glycero-3-phosphocholine). In some embodiments, the phosphocholine is used in an amount of about 1-3 mg per 75 μL of perfluorocarbon. In some embodiments, the coating material comprises cholesterol, e.g., in an amount of about 1 mg per 75 μL of perfluorocarbon. In some embodiments, the coating material comprises a polymer.

In certain embodiments the nanocarriers comprise a Zonyl FSO fluorosurfactant shell and perfluoropentane (boiling point=29° C.) core.

In certain embodiments, the nanoparticles are dispersed within the perfluorocarbon solution. In certain embodiments, the nanoparticles are on the surface of the perfluorocarbon solution (e.g., on the edge between the shell).

In some embodiments, the nanocarrier of the present invention, has an exterior diameter (e.g., a hydrodynamic diameter) of about 50 nm to about 500 nm, or about 100 nm to about 500 nm. In some embodiments, the mean hydrodynamic diameter of the nanocarrier is about 100 nm to about 300 nm or about 200 nm or about 100 nm. In one specific embodiment, the mean hydrodynamic diameter of the nanocarrier is about 220 nm, or about 250 nm. In some embodiments, the nanocarriers of the invention are in the form of a micelle-like structure (as distinguished from liposome structures comprising a lipid bilayer), being approximately spherical in shape, although forms, including ellipsoids, cylinders and other shapes are also possible. Without being bound by any theory of invention, it is believed that the size (e.g., about 100 nm to about 300 nm) and shape (e.g. a micelle structure) of the nanocarriers of the invention impart several advantages to the nanocarrier compositions. For example, the nanocarriers of the invention are believed to extravasate diseased vasculature and into tissue (e.g. within inflamed tissue and/or within cancerous tissue) for targeted diagnostic and therapeutic applications. The organic components of the nanocarrier are largely biodegradable and can be cleared in a timely fashion. The inorganic components of the nanocarrier, i.e., copper sulfide nanoparticles, can be synthesized at a size that will readily pass through the renal system and be rapidly cleared while still possessing strong absorbance in the extended NIR.

In some embodiments, the nanocarrier composition may further comprise a therapeutic agent. The therapeutic agent may be included in the organic liquid or core of the nanocarrier or on the surface thereof, for example, attached to or within the coating material.

In some embodiments, the therapeutic agent may be an anti-inflammatory agent. Suitable anti-inflammatory agents include those that reduce inflammation or swelling in a subject (e.g., caused by infection, injury, irritation, or surgery). In certain embodiments, the anti-inflammatory agent may include a steroidal or non-steroidal anti-inflammatory agent. Steroidal anti-inflammatory agents include, for example, dexamethasone and betamethasone. Non-steroidal anti-inflammatory agents may COX-1 or COX-2 inhibitors, such as diclofenac, piroxicam or indomethacin.

In certain embodiments, the therapeutic agent may be an anti-cancer agent. Any suitable anti-cancer agent may be used in the compositions and methods of the present disclosure, including agents which can be used to treat a cell proliferative disorder, such as, cytotoxic agents, cytostatic agents, anti-angiogenic agents, debulking agents, chemotherapeutic agents, radiotherapy and radiotherapeutic agents, targeted anti-cancer agents, biological response modifiers, therapeutic antibodies, cancer vaccines, cytokines, hormone therapy, anti-metastatic agents and immunotherapeutic agents. The selection of a suitable anti-cancer agent may depend upon, among other things, the type of cancer to be treated and the composition of the nanocarrier compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. In certain embodiments, the anti-cancer agent may be, but is not limited to, gemcitabine, doxorubicin, 5-fluorouracil, or paclitaxel.

In certain embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions and methods of the present disclosure, among other things, may increase the concentration of the anti-cancer agent in the compositions and methods of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In some embodiments, the nanocarrier composition of the present invention may comprise a targeting moiety. The targeting moieties useful in the compositions and methods of the present invention include molecules that may be bound to an exterior surface of a nanocarrier composition and which recognize a particular site of interest in a subject. In certain embodiments, the targeting moieties may be bound directly to the coating material or bound to the coating material using a linking molecule.

In certain embodiments, the targeting moiety may be chosen, among other things, to at least partially increase the uptake of the compositions of the present disclosure into a desired cell and/or tissue type when introduced into a subject. In certain embodiments, the targeting moiety may recognize a particular ligand or receptor present in a desired cell and/or tissue type when introduced into a subject. In certain embodiments, the targeting moiety may be an antibody that recognizes such a particular ligand or receptor. The use of antibody fragments and bispecific antibodies may also be suitable in the compositions and methods of the present disclosure. The choice of a targeting moiety may depend upon, among other things, the cell and/or tissue type into which an at least partial increase in uptake of the compositions of the present disclosure is desired, as well as particular ligand(s) present in such cell and/or tissue types. In certain embodiments, the targeting moiety may be a moiety that recognizes a molecule which is present in higher amounts in an abnormal form of a tissue when compared to a normal form of the same tissue (i.e., the molecule is “up-regulated” in the abnormal form of the tissue). For example, in certain embodiments, antibodies which bind to epidermal growth factor (EGFR) may be suitable for use in the compositions and methods of the present disclosure when it is desired to at least partially increase the uptake of the compositions of the present disclosure into cancerous epithelial tissue. As a further example, antibodies such as anti-Claudin-4, anti-Mucl, anti-HER2, or anti-EGFR may be suitable for use in the compositions and methods of the present disclosure when it is desired to at least partially increase the uptake of the compositions of the present disclosure into cancerous pancreatic tissue. In some embodiments, a suitable targeting moiety may be a peptide sequence, DNA fragment, aptamer, RNA, folate, polymer, etc. One of ordinary skill in the art, with the benefit of this disclosure, will recognize other targeting moieties that may be useful in the compositions and methods of the present disclosure. Such targeting moieties are considered to be within the spirit of the present disclosure. In some embodiments, the targeting agent comprises EGFR and HER2, or VEGFR-based targeting moieties.

In certain embodiments, the targeting moieties useful in the compositions and methods of the present disclosure may be bound directly to the coating material. In certain embodiments, the targeting moieties useful in the compositions and methods of the present disclosure may be bound to the coating material via a linking molecule. The linking molecules useful in the compositions and methods of the present disclosure may be any molecule capable of binding to both the coating material used in the compositions and methods of the present disclosure and the targeting moieties used in the compositions and methods of the present disclosure. In certain embodiments, the linking molecule may be a hydrophilic polymer. Suitable linking molecules include, but are not limited to, poly(ethylene glycol) and its derivatives, dithiol compounds, dithiol compounds with hydrazide and/or carboxylic functionality, or single thiols and/or amines or their derivatives. In certain embodiments, the linking molecule and the targeting moiety may be bound by one or more covalent bonds. In certain embodiments, the linking molecule, in addition to linking the targeting moiety and the coating material, may impart certain benefits upon the compositions of the present disclosure, including, but not limited to, improved hydrophilicity and stability in solution, reduced immunogenic responses upon introduction of the compositions of the present disclosure into a subject, increased circulation time of the compositions of the present disclosure when introduced into the bloodstream of a subject. The choice of a linking molecule may depend upon, among other things, the targeting moiety chosen and the subject into which the compositions of the present invention are to be introduced. One of ordinary skill in the art, with the benefit of this disclosure, will recognize additional suitable linking molecules. Such linking molecules are considered to be within the spirit of the present disclosure.

In some embodiments, nanocarrier compositions of the present disclosure may further comprise gene components, such as siRNA or therapeutic DNA fragments. In some embodiments, a gene component may be included in the organic liquid of the nanocarrier compositions. In some embodiments, a gene component may be on the surface of the nanocarrier composition, for example, attached to or within the coating material. These agents may be used for gene therapy or to enhance sensitivity in drug resistant cell lines.

In some embodiments, the nanocarrier agents of the invention are provided in the form of an aqueous composition. In certain embodiments, the composition may comprise an aqueous layer comprising a plurality of nanocarriers of the invention dispersed therein. In some embodiments, the nanocarrier agents in the aqueous layer are substantially monodisperse within the aqueous layer, e.g., having a polydispersity index of less than about 0.15 or less than about 0.1. In certain embodiments, the aqueous layer comprises a saline solution (such as phosphate buffered saline).

In some embodiments, the carrier agent comprises a perfluorocarbon solution (e.g., perfluoropentane) comprising a plurality of fluorinated copper sulfide nanoparticles dispersed therein, and a fluorinated surfactant coating material disposed around the exterior surface of the perfluorocarbon solution. The advantageous utility of copper sulfide nanoparticles in some embodiments was unexpected. Copper sulfide nanoparticles are generally thought to be of limited utility for imaging applications as they only offer photoacoustic contrast and often require high concentrations to provide sufficient signal to noise. However, it has been discovered that when copper sulfide nanoparticles are combined with perfluorocarbons, as described herein, enhanced photoacoustic and ultrasound contrast can be achieved such that there is improved diagnostics as well as favorable clinical characteristics associated with copper sulfide nanoparticles. Use of copper sulfide nanoparticles as nanodroplet carriers further allows for controlled droplet vaporization within the second optical imaging window for increased penetration, enhanced ultrasound and photoacoustic contrast.

### B. Synthesis

The present invention also provides methods for preparing nanocarriers of the invention. In certain embodiments, the nanocarriers can be prepared by: providing a nanoparticle precursor, fluorinating the nanoparticle precursor, contacting the fluorinated nanoparticle with a perfluorocarbon solution and a coating material to afford a nanocarrier agent.

In some embodiments, the carriers of the invention are prepared by first synthesizing a nanoparticle precursor. The nanoparticle precursors can include coated and uncoated nanoparticles such as those described previously (Zhou, M. et al., A chelator-free multifunctional [64Cu] CuS nanoparticle platform for simultaneous micro-PET/CT imaging and photothermal ablation therapy. Journal of the American Chemical Society 2010, 132 (43), 15351-15358.). In certain embodiments, the nanoparticle precursor is a copper sulfide nanoparticle. In one specific embodiment, the nanoparticle precursor is a citrate-coated (or citrate-stabilized) copper sulfide nanoparticle.

Next, the nanoparticle precursor can be coated to facilitate its encapsulation in the nanoparticle carrier of the invention. In some embodiments, the coating involves fluorinating the nanoparticle precursor to afford a fluorinated nanoparticle. In certain embodiments, the coating is a two-step process in which the nanoparticle precursor (e.g., a citrated-coated or citrate-stabilized nanoparticle) is first PEGylated to afford a PEGylated nanoparticle. The PEGylated nanoparticle is then fluorinated to afford a fluorinated nanoparticle. The fluorination is believed to ensure solubility of the nanoparticles in certain organic liquids, such as perfluorocarbons. In certain embodiments, a fluorinated copper sulfide nanoparticle is prepared by way of this method. As copper sulfide nanoparticle absorbance depends on d-d transition, fluorination of copper sulfide is not believed to significantly alter its structure, characteristic UV-Vis spectrum or ability to absorb.

In some embodiments, the coated (or fluorinated) nanoparticle can be combined with an organic liquid (such as a perfluorocarbon) and a coating material (such as a fluorosurfactant) to afford a nanocarrier of the invention. In certain embodiments, a nanocarrier of the present invention may be synthesized through an “organic liquid in water” emulsion. For example, a coating material, such as a surfactant or protein, may be dissolved in water and aqueous nanoparticles may undergo a process of phase transfer. The phase transferred nanoparticles may then be dispersed into the organic liquid.

### C. Methods of Use

The nanocarrier compositions of the present invention can be used in numerous areas including, but not limited to, imaging, drug delivery, diagnostics, and therapy.

In one embodiment, the present invention provides a method of imaging comprising providing a nanocarrier composition comprising: an organic liquid comprising a plurality of nanoparticles dispersed therein, and a coating material disposed around the exterior surface of the organic liquid; and imaging a biological tissue comprising the nanocarrier composition. In another embodiment, the present disclosure provides a therapeutic method comprising contacting a biological tissue with a nanocarrier composition comprising: an organic liquid comprising a plurality of nanoparticles dispersed therein, a coating material disposed around the exterior surface of the organic liquid, and a therapeutic agent.

In some embodiments, the nanocarrier compositions of the present disclosure may act as a contrast agent for continuous wave photoacoustic imaging, combined photoacoustic and ultrasound imaging, magnetomotive imaging, optical coherent tomography, magnetic resonance imaging, computed tomography, nuclear imaging modalities or any combination thereof. Furthermore, when the nanocarriers contain magnetic iron oxide and/or cobalt nanoparticles, they may be used in microwave ablation therapy and magnetomotive imaging enhancement. In one embodiment, nanoparticles dispersed within the organic liquid may absorb light energy typically employed during photoacoustic imaging techniques. Therefore, the nanoparticles may act in their traditional role as photoacoustic contrast agents. Simultaneously, the absorption of that light energy by the nanoparticles may cause them to heat, thereby “activating” the organic liquid as an ultrasound contrast agent, for example, by vaporizing the organic liquid. This activation may create an impedance mismatch between the organic gas (from the vaporized organic liquid) and the surrounding blood and tissues, providing strong ultrasound imaging contrast. Therefore, the nanocarrier compositions may act as a contrast agent for photoacoustic imaging at the cellular level in two ways: (1) absorption from the nanoparticles, and (2) extra induced pressure waves generated by the vaporization of the organic liquid.

In some embodiments, energy such as an electromagnetic field, optical methods, or specific radiofrequencies may be applied to biological tissue thereby causing the vaporization of the organic liquid and if the nanocarrier further comprises a therapeutic agent, the release of the therapeutic agent. In some embodiments, this may provide a clinician the ability to control and visualize drug therapy noninvasively.

Some embodiments of the present invention provide methods of using nanocarriers to detect the size and proper boundaries of tumor regions. In certain embodiments, nanocarrier compositions of the present disclosure may be delivered to cancerous tissue. Delivery methods may include patient injection of nanocarriers, and may also include using targeting moieties to help facilitate accumulation in a diseased tissue. It is believed that this method may provide two or more mechanisms of enhancing diagnostic imaging contrast. When used in conjunction with a combined photoacoustic and ultrasound imaging system, the nanocarriers may strengthen photoacoustic signals from the tumor region while simultaneously increasing ultrasound contrast. When iron oxide nanoparticles are included in the nanocarriers, magnetic resonance imaging, and photoacoustic and/or ultrasound imaging may be used in conjunction. Therefore, two or more imaging modalities may be used by clinicians to verify the location and size of diseased tissue by using a simple injection of nanocarriers.

Additionally, according to embodiments of the present invention, nanocarriers comprising both therapeutic agents and targeting moieties may act as a targeted delivery system for therapeutic agents.

Some embodiments provide methods for the use of the organic gas bubbles as vascular blocking agents to initiate necrosis in a specific location of tissue (e.g. blocking tumor vasculature). In some embodiments, deposition of nanocarriers at the region of necrosis would permit photoacoustical monitoring of the decay.

The invention will be described in greater detail by way of specific examples. The following examples are offered for illustrative purposes and are not intended to limit the invention in any manner. Those of skill in the art will readily recognize a variety of noncritical parameters which can be changed or modified to yield essentially the same results.

### EXAMPLES

Reagents and solvents used below can be obtained from commercial sources such as Sigma-Aldrich. All imaging experiments were conducted using a 40-MHz ultrasound and photoacoustic imaging probe (LZ-550, Visualsonics Inc) coupled to a combined ultrasound and photoacoustic VevoLAZR imaging system (Vevo 21100, Visualsonics Inc). Samples were irradiated with a laser beam using 5-7 ns pulses and 6-10 mJ/cm² fluence.

### Example 1

### Preparation of Fluorinated Copper Sulfide Nanoparticles

This example describes the synthesis of fluorinated copper sulfide nanoparticles.

Citrate-coated copper sulfide nanoparticles were synthesized according to previously described methods (Zhou, M. et al., A chelator-free multifunctional [64Cu] CuS nanoparticle platform for simultaneous micro-PET/CT imaging and photothermal ablation therapy. Journal of the American Chemical Society 2010, 132 (43), 15351-15358.). The hydrodynamic size of the nanoparticles was measured on a Zetasizer (Malvern) and shown to be ˜11 nm, which agreed with TEM images. The citrate-coated copper sulfide nanoparticles were PEGylated via addition of thiolated poly(ethylene glycol) (mPEG-SH, 5 KDa Layson Bio) (1 mg/ml in DI water) solution of equal volume. The solution was left to react overnight under gentle stirring. The solution was then washed of excess PEG using 30 kDa Amicon centrifuge filters and resuspended in water at an O.D. of −10 (O.D. was tested at a range of wavelengths around the peak (e.g., 700-1100 nm) and peak value was used for O.D. measurement).

The PEGylated nanoparticles were then fluorinated using previously described methods. Specifically, a solution of PEGylated copper sulfide nanoparticles (3.5 ml at OD of −10) was added to ethanol (20 ml) and 1H, 1H, 2H,2H-Perfluorodecanethiol (Sigma) (300 μl), and the mixture was stirred and left to react overnight. After 24 hours, the resulting fluorinated copper sulfide nanoparticles were washed in Millipore at 1000 rcf for 10 minutes. The remaining solution, <1 ml, was placed under reduced pressure (Bunchi Rotavapor®) to remove any remaining ethanol or excess perflourodecanethiol producing a copper sulfide cake. Next, depending on nanocarrier synthesis and timing, either phosphate buffered saline (PBS) (1 ml) or chloroform (1 mL) was added to the dried fluorinated copper sulfide nanoparticle and sonicated until the nanoparticles became resuspended. Slight nanoparticle aggregation is typical especially when resuspended in PBS.

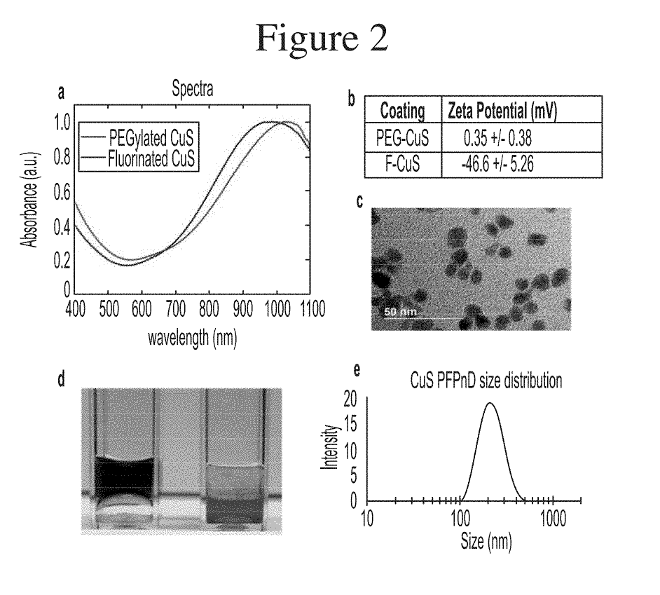

The PEGylated and fluorinated copper sulfide nanoparticle solutions were analyzed on an Evolution 220 UVvis Spectrophotometer (Thermo Fisher). Zeta Potential measurements of the solutions were performed on a Zetasizer Nano ZS (Malvern). As shown in FIG. 2(a), fluorination of the copper sulfide nanoparticles did not significantly alter their absorbance spectrum. The fluorinated copper sulfide nanoparticles were found to have appropriate size and structure by TEM (FIG. 2[c]). Zeta potential measurements confirmed successful coating of copper sulfide nanoparticles at each step (FIG. 2[b]).

### Example 2

### Nanoparticle Stability

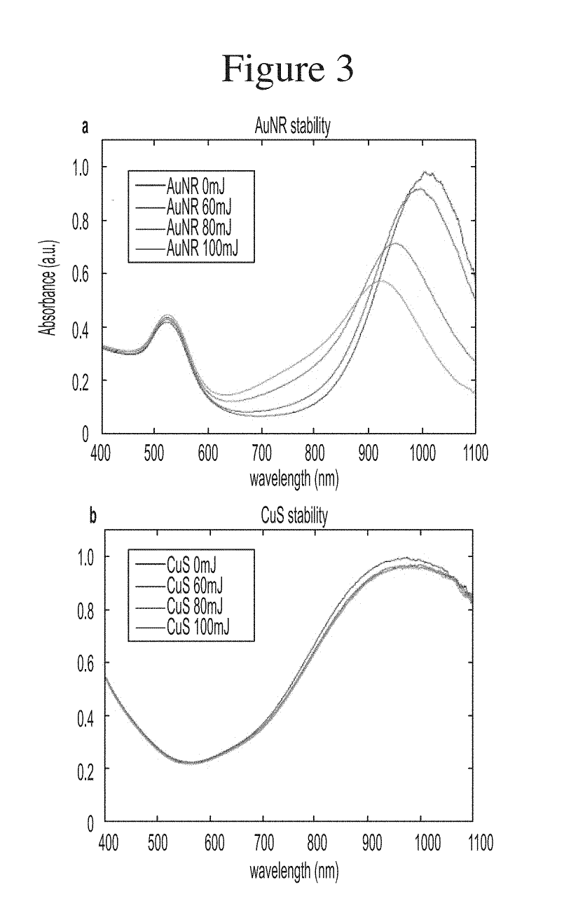

In this experiment, the stability of gold nanorods and copper sulfide nanoparticles was tested. The copper sulfide nanoparticles tested in this experiment are those prepared in Example 1 (PEG-coated and suspended in PBS). Solutions of gold nanorods and copper sulfide nanoparticles of equivalent optical densities were irradiated at different energies (number of pulses=600). Laser irradiation had an effect on the gold nanorod stability, which, in turn, affected the resulting spectrum and its ability to absorb at 1064 nm (FIG. 3[a]). The copper sulfide nanoparticle spectrum was not altered by laser irradiation, even at the highest energies, demonstrating the photothermal stability of the copper sulfide nanoparticles (FIG. 3[b]).

### Example 3

### Preparation of Nanocarrier

Perfluoropentane (50 μl; FluoroMed L.P.) and Zonyl FSO fluorosurfactant (150 ul of 1% v/v; Sigma) were added to the resuspended fluorinated copper sulfide nanoparticle solution prepared in Example 1. As shown in FIG. 2(d), the fluorinated copper sulfide nanoparticles were found to be soluble within the perfluorocarbon solution while the PEGylated copper sulfide nanoparticles remained in the PBS layer. The solution was vortexed for 10 s and sonicated in an ice-cold ultrasonic water bath (VWR, 180 W) for 5 minutes or until the solution became milky and minimal excess perfluoropentane was observed. The resulting mixture was then centrifuged at 100 rcf for 1 minute to removes any excess copper sulfide that might have aggregated and remained unencapsulated in the nanocarrier. The supernatant was taken and further washed at 1000 rcf for 5 minutes to isolate the nanocarriers. After resuspending in PBS, the hydrodynamic radius of the nanocarrier composition was measured using DLS (characterized with a Zetasizer Nano ZS) shortly after sample preparation. As shown in FIG. 2(e), the synthesized nanocarriers were monodisperse with an average size of 220 nm.

### Example 4

### Phantom Imaging

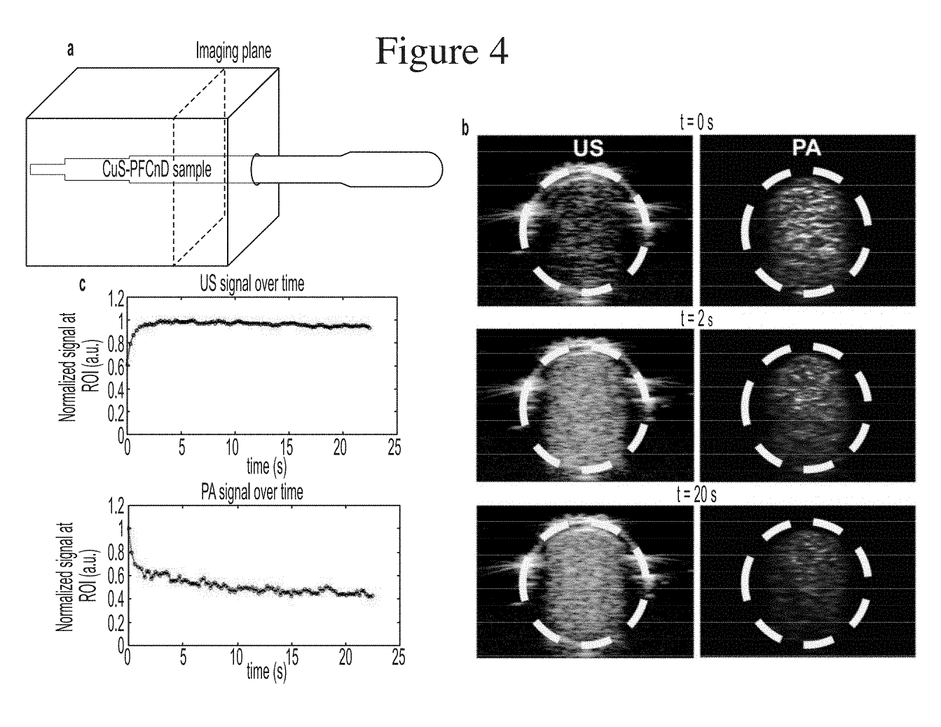

In this experiment, the photoacoustic and ultrasound properties of the nanocarrier composition prepared in Example 3 (Zonyl FSO shell) was tested in a tissue model. A poly(acrylamide) tissue mimicking phantom with a pipette inclusion was constructed as previously described (Hannah, A. et al., Indocyanine green-loaded photoacoustic nanodroplets: dual contrast nanoconstructs for enhanced photoacoustic and ultrasound imaging. ACS nano, 2013, 8 (1), 250-259). The nanocarrier composition prepared in Example 3 was diluted (2.0% v/v) in PBS. A pipette containing the diluted solution (concentration 1×10⁶ nDs/ml) was placed in the phantom. The phantom used in this experiment is illustrated in FIG. 4(a) (the 2-D imaging plane is marked by a dashed square). Ultrasound gel was placed on the top of the phantom, and the imaging probe positioned so that the optical focus was at the pipette intersection. A Vevo 2100/LAZR system was used to capture ultrasound and photoacoustic information. 1064 nm nanosecond (5-7 ns) pulsed laser was used to irradiate the sample inclusion. Ultrasound B-mode and photoacoustic (1064 nm) were collected simultaneously for 300 frames. Fluences of 6-10 mJ/cm², which is 10% of the permitted ANSI fluence at this wavelength, were enough to activate the nanocarriers. After the first laser pulse, the majority of nanocarriers were vaporized, resulting in a strong photoacoustic signal and the formation of microbubbles (FIG. 4[b]). As the irradiated nanocarriers became stable microbubbles, ultrasound contrast became enhanced. Subsequent laser pulses activated remnant or new nanocarriers that flowed into the plane; however, the majority of the photoacoustic signal observed at these time points came from copper sulfide nanoparticles, which were either entrapped within the created microbubbles or had escaped during the vaporization process. Changes in photoacoustic and ultrasound signals were plotted over time for comparison (FIG. 4[c]). Quantitative analysis revealed that the ultrasound contrast rapidly increases as stable microbubbles are formed. In contrast, photoacoustic signal is at maximum upon nanocarrier vaporization and subsequently decays, eventually plateauing to a stable photoacoustic signal from copper sulfide nanoparticles (FIG. 4[c]).

### Example 5

### In Vivo Imaging of Organs and Ultrasound Image Processing of Nanocarriers

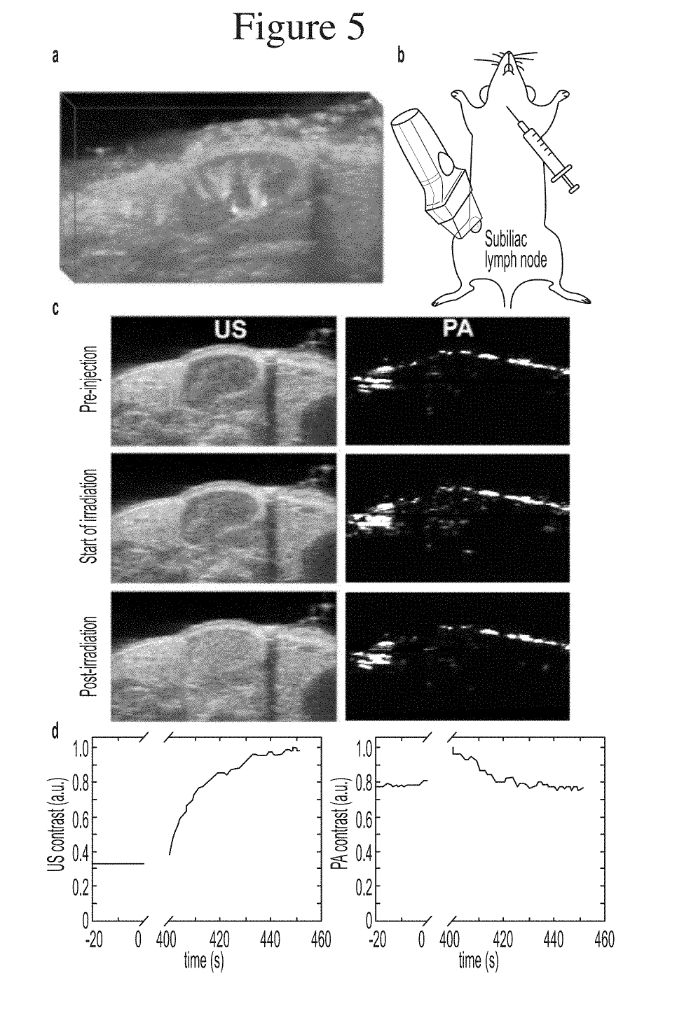

In this experiment, the nanocarrier composition prepared in Example 3 was tested in vivo. The in vivo imaging studies described here adhered to approved protocols by the Institutional Animal Care and Use Committee at Georgia Institute of Technology. In this study, a mouse exhibiting signs of dermatitis was used. As an inflammatory disease, dermatitis leads to certain morphological changes, such as lymphangiogenesis and vasculature hyperpermeability that mimic premetastatic lymph nodes. (Huggenberger, R. et al, In The cutaneous vascular system in chronic skin inflammation, Journal of Investigative Dermatology Symposium Proceedings, Elsevier: 2011, pp 24-32; Mumprecht, V. et al., Inflammation-induced lymph node lymphangiogenesis is reversible. The American journal of pathology 2012, 180 (3), 874-879; Varricchi, G. et al., Angiogenesis and lymphangiogenesis in inflammatory skin disorders. Journal of the American Academy of Dermatology 2015, 73 (1), 144-153; Coussens, L. M. et al., Inflammation and cancer. Nature 2002, 420 (6917), 860-867). Therefore, the model presented in this experiment is also highly relevant to use of the nanocarrier compositions in cancer applications. The mouse was anesthetized using a combination of isofluorane (1.5-2.5%) and O₂ (1 L/min). The subiliac lymph node, in the inguinal region, was localized and ultrasound and Doppler images were acquired (FIG. 5[a]). As shown in FIG. 5(a), 3-D Doppler ultrasound showed an enlarged lymph node and vasculature, characteristics of inflammatory diseases. The copper sulfide nanocarriers prepared in Example 3 (150 μL) were injected intravenously through the jugular vein (FIG. 5[b]). Ultrasound images were taken to ensure there was no spontaneous vaporization of the copper sulfide nanocarriers that would be mistaken as laser-activated nanocarriers. No microbubble appearance or spontaneous formation was observed, indicating that the nanocarriers were stable in vivo. Two minutes after injection the sample was irradiated at 1064 nm and ultrasound/photoacoustic signal was collected. An increase in ultrasound and photoacoustic signal was observed (FIG. 5[c]). After the initial laser pulse, there was an intense photoacoustic signal that decayed over time, coupled with a rapid increase in ultrasound signal that was similar to the signal observed in the tissue model (FIG. 5[d]). This similarity suggests that a portion of the nanocarriers remain within the 2-D imaging plane, becoming trapped in the lymph node either within capillaries or lymph tissue. Given that the vasculature of an inflamed lymph node becomes hyperpermeable, it is likely a subset of the nanocarriers were able to extravasate. This is supported by the ultrasound enhancement seen throughout the entirety of the lymph node (FIG. 5[d]).

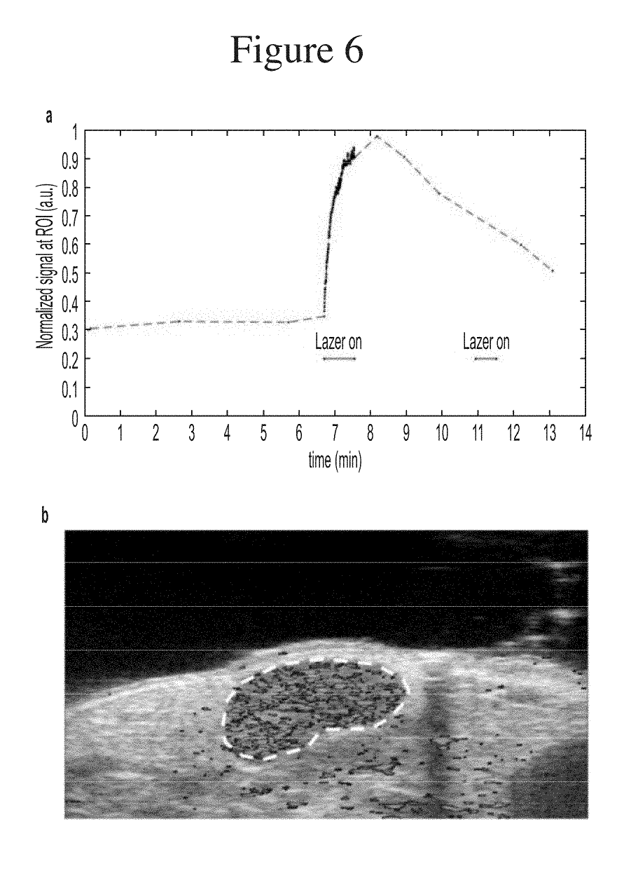

The ultrasound contrast throughout the lymph node persisted for several minutes, gradually decaying over time. To better characterize the contrast, linear regression was used to calculate the slope of each pixel's ultrasound signal during the first laser irradiation period. Pixels with signals that exhibit a slope above a certain threshold are identified as locations where microbubbles were created, i.e. nanocarrier phase-change occurred. The selected pixels are displayed over an ultrasound image for anatomical reference, resulting in an ultrasound-based background-free image with a clear visualization of the node as well as surrounding vasculature (FIG. 6[b]). As shown in FIG. 6(a), the average ultrasound contrast inside the region (lymph node) of interest increased upon laser irradiation and gradually decayed as the bubbles diffuse and disappear from the field of view. In FIG. 6(b), the overlaid contrast image shows pixels with vaporized nanocarriers, identified by a high slope in ultrasound signal during laser irradiation. The majority of vaporized nanocarriers were located within the lymph node (white dashed outline), but nanocarriers were also present in surrounding vasculature.

The ability of systemically injected nanocarriers to dramatically enhance image contrast of an inflamed lymph node presents several opportunities. Morphological changes experienced during inflammatory diseases closely resemble changes seen in pre-metastatic lymph nodes. Therefore, this experiment demonstrates that nanocarriers can be used to uncover diseased and hyperpermeable lymph node vasculature. Additionally, intravenous injections overcome the limitation associated with subcutaneous injection of larger particles (>100 nm), where the majority of particles remain entrapped at the injection site and are unable to enter the lymphatics (Reddy, S. et al., In vivo targeting of dendritic cells in lymph nodes with poly (propylene sulfide) nanoparticles. Journal of Controlled Release, 2006, 112 (1), 26-34; Oussoren, C. et al., Liposomes to target the lymphatics by subcutaneous administration. Advanced Drug Delivery Reviews, 2001, 50 (1), 143-156; Zhang, F. et al., Preclinical lymphatic imaging. Molecular Imaging and Biology, 2011, 13 (4), 599-612).

This experiment demonstrates that the nanocarriers have potential applications in other diseases as well, such as infections, where the droplets can be used to deliver therapeutics to infected lymph nodes, in a highly specific, on-demand, manner.

The nanocarriers may also be used as photoabsorbers. The nanocarriers provide photoacoustic contrast as deep as 5 centimeters due to their extended NIR absorbance (Ku, G. et al., Copper sulfide nanoparticles as a new class of photoacoustic contrast agent for deep tissue imaging at 1064 nm, Acs Nano, 2012, 6 (8), 7489-7496). This signifies that the nanocarriers can be activated at increased depths compared to other photoabsorbers, allowing for comprehensive imaging at clinically relevant depths. Furthermore, the nanocarriers have shown biodegradability in vivo, overcoming concerns of long-term biocompatibility that other inorganic particles, such as gold nanoparticles, face (Guo, L. et al., A comparative study of hollow copper sulfide nanoparticles and hollow gold nanospheres on degradability and toxicity, ACS nano, 2013, 7 (10), 8780-8793).

The particles can also be synthesized at sizes that pass through the renal system for more rapid clearance, and still possess absorbance in the extended NIR (Zhou, M. et al., CuS nanodots with ultrahigh efficient renal clearance for positron emission tomography imaging and image-guided photothermal therapy. ACS nano, 2015, 9 (7), 7085-7096; Cheng, Z. et al., Facile fabrication of ultrasmall and uniform copper nanoparticles, Materials Letters, 2011, 65 (19), 3005-3008).

The photothermal stability of the nanocarriers makes them ideal for imaging transient microbubbles, which require persistent pulsed irradiation (Luke, G. P. et al., Super-resolution ultrasound imaging in vivo with transient laser-activated nanocarriers, Nano letters, 2016, 16 (4), 2556-2559).

Copper sulfide nanoparticles can also be used as photothermal agents. Therefore, in addition to augmenting ultrasound/photoacoustic contrast, the nanocarriers have inherent characteristics for use as therapeutic agents. Overall, the use of copper sulfide nanoparticles to trigger nanocarrier vaporization brings laser-activated nanocarriers closer to clinical relevance by possessing biocompatibility and degradability, and providing superior, clinically-relevant diagnostic characteristics.

### Example 6

### Stability of Nanocarriers In Vivo and Photoacoustic Image Processing of Nanocarriers

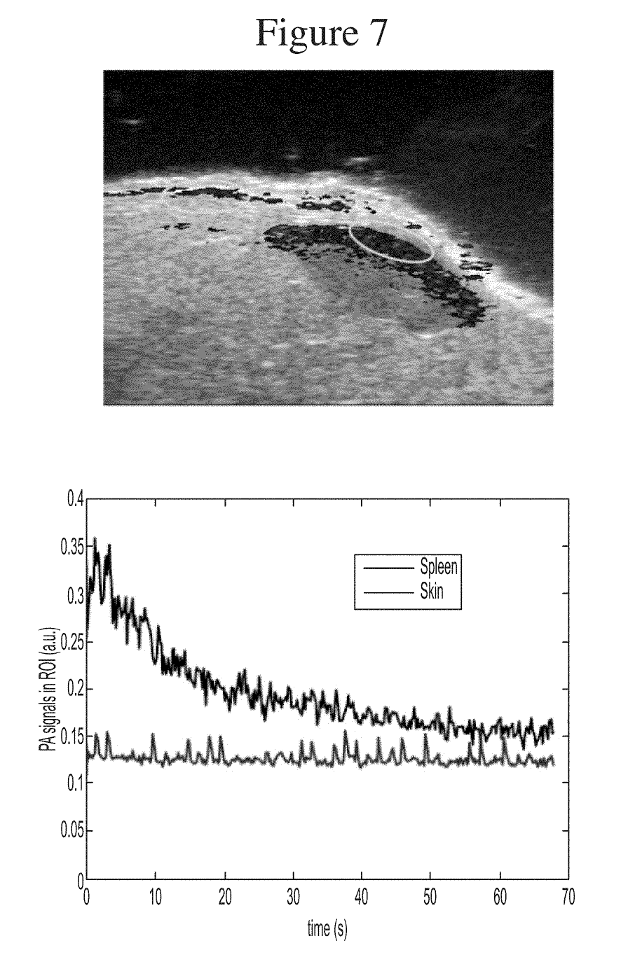

The mouse model procedure described in Example 5 was used in this experiment. FIG. 7(a) shows an ultrasound/photoacoustic image of the spleen (dashed outline) upon initial irradiation at 1064 nm, one hour after injection. The changes in photoacoustic signal of ROIs representing the spleen and skin shown in FIG. 7(b) indicated the presence of activated nanodroplets. After ultrasound contrast returned to baseline, the spleen of the mouse was imaged to study the copper sulfide nanocarrier accumulation in organs. Ultrasound images did not show any obvious microbubbles, but after irradiating and acquiring ultrasound/photoacoustic signal a spike in photoacoustic signal was observed that exponentially decayed, indicative of vaporizing of the nanocarriers. Hence, a portion of the copper sulfide nanocarriers are trafficked to the spleen where they remain stable nanodroplets for at least one hour, upon which they can be activated via laser irradiation.

To localize vaporized nanocarriers based on photoacoustic signal, pixels with a rapidly decreasing photoacoustic signal were identified via image processing techniques. For the technique to effectively work, it is important to image for a significant time frame in order to capture both the decay and baseline nanocarrier temporal pattern. Then, frames that capture the decay are selected, avoiding frames that depict photoabsorber/nanoparticle baseline signal (i.e. steady state signals), and pixel-wise linear regression is performed. Prior to the linear fit, undesired sample motion can be accounted for by applying a spatial averaging window in spatial (X-Y plane) and time (Z) dimension. Once the linear fit is determined, the slopes from each pixel are extracted. The slopes indicate the change in photoacoustic signal over the studied time, therefore higher value slopes correlate to vaporized nanocarriers, while those with a slope closer to 0 indicate endogenous photoacoustic signal or noise. A threshold value is selected to only display pixels that exhibit a large variation rate, i.e. indicative of vaporized nanocarriers. The identified pixels are then displayed over an ultrasound image for anatomical reference, resulting in a photoacoustic-based background-free image (FIG. 7).

### Example 7

### Preparation of Nanocarrier

To synthesize the nanocarrier with a lipid shell two methods were employed. The first method consisted of making a lipid cake by combining 1,2-distearoyl-sn-glycero-3-phosphocholine (DSPC, 10 mg/mL; NanoCS Inc.) and 1,2-distearoyl-sn-glycero-3-phosphoethanolamine-N-[amino(polyethylene glycol)-2000] (DSPE-PEG(2000), 25 mg/ml; Avanti Polar Lipids, Inc.), both in chloroform, to a pear shaped flask. An additional 2 ml of chloroform were added to the flask to facilitate the production of a smooth lipid cake. The flask was placed under reduced pressure (Rotavapor; BUCHI Labortechnik) to remove the chloroform, and the result was a thin, lipid cake. The lipid cake was hydrated with PBS, transferred to a scintillation vial and sonicated to produce micelle structures. Then, fluorinated copper sulfide nanoparticles resuspended in PBS (from Example 1) and perfluoropentane were added to the vial and nanocarriers were synthesized through sonication in ice cold water. Sonication was stopped when there was no significant perfluoropentane bolus. A similar centrifugation described for the Zonyl shell was employed to clean and then characterize the lipid nanocarriers.

The second method of synthesis involved adding fluorinated copper sulfide nanoparticles resuspended in chloroform (from Example 1) to the lipid cake synthesis step. Specifically, a volume of fluorinated copper sulfide in chloroform was added to the DSPC, DSPE-PEG(2000) and excess chloroform solution. The solution, contained in a pear shaped flask, was placed under reduced pressure (Rotavapor; BUCHI Labortechnik) to remove the chloroform and produce a thin, blue-colored lipid cake. Next, PBS was added to rehydrate the copper sulfide/lipid cake (sonicated if needed). The solution was then transferred to a scintillation vial and perfluoropentane was added. Lipid nanocarriers were produced by sonicating the mixture in ice cold water until no significant perfluoropentane bolus remained. A similar centrifugation described for the Zonyl shell was employed to characterize the lipid nanocarriers.

### Example 8

### Producing Photoacoustic-Based Background-Free Images

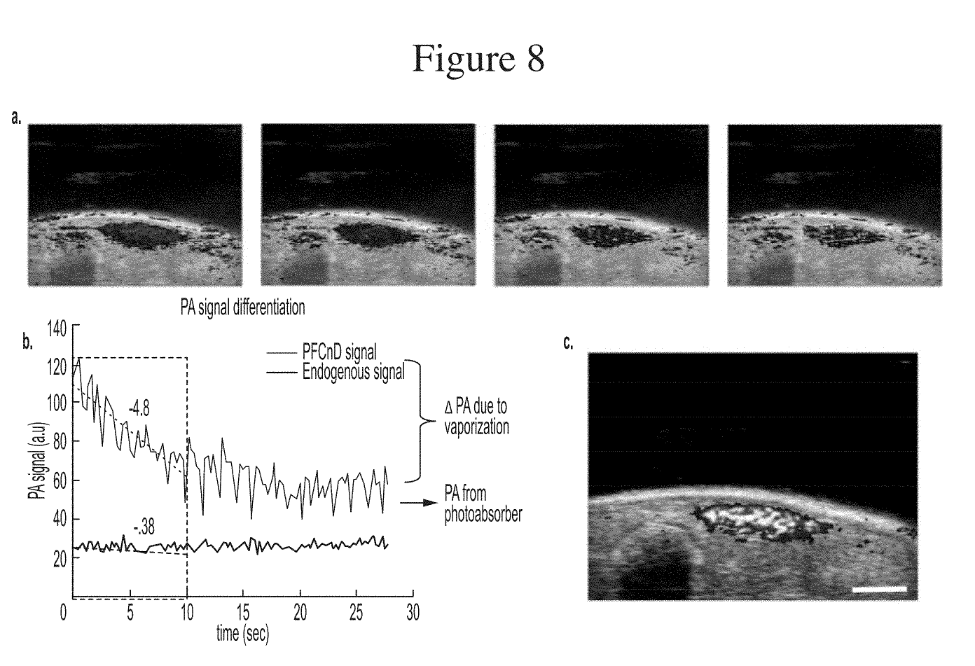

Upon laser induced perfluorocarbon nanocarrier vaporization, there is an intense photoacoustic signal. In stationary samples, the signal is at a maximum immediately after the laser pulse that causes vaporization, which is typically the first laser pulse, and then decreases over time (FIG. 8). The rate at which the signal decreases is dependent on the perfluorocarbon nanocarrier portion that is vaporized compared to the total that are vaporized throughout the imaging period. If the entire perfluorocarbon nanocarrier population in the region of interest is vaporized after the first laser pulse, then the maximum photoacoustic is large and the drop in photoacoustic signal is drastic. If some unvaporized perfluorocarbon nanocarriers remain, or subsequently enter the image plane, and are vaporized on ensuing laser pulses, then the peak photoacoustic signal is reduced, and the decay is more gradual. When all vaporization events have occurred, the photoacoustic signal drops to a baseline level representative of the thermoelastic expansion of the photoabsorber utilized (FIG. 8[b]). The difference between the maximum photoacoustic signal and the baseline photoacoustic signal is referred to as vaporization-associated photoacoustic signal. The larger the difference, the more effective the photoabsorber is at initiating perfluorocarbon nanocarrier vaporization.

Background-free imaging through localization of perfluorocarbon nanocarrier is extremely advantageous because of the endogenous photoacoustic signal seen at many wavelengths. Even at 1064 nm, which is within the optical window, there is still endogenous signal (FIG. 8[a]). To localize vaporized perfluorocarbon nanocarriers, pixels with a rapidly decreasing photoacoustic signal were identified via image processing using MATLAB. For the technique to effectively work, it is important to image for a significant time frame in order to capture both the decay and baseline perfluorocarbon nanocarrier temporal pattern. Then, frames that capture the decay, but avoid frames depicting photoabsorber baseline signal, are selected (FIG. 8(b), dashed square) and pixel-wise linear regression is performed. Prior to the linear fit, undesired sample motion can be accounted for by applying a spatial averaging window in spatial (X-Y plane) and time (Z) dimension. Once the linear fit is determined, the slopes from each pixel are extracted. The slopes indicate the change in photoacoustic signal over the studied time, therefore higher value slopes correlate to vaporized perfluorocarbon nanocarriers, while those with a slope closer to 0 indicate endogenous photoacoustic signal or noise (FIG. 8[b]). A threshold value is selected to only display pixels that exhibit a large variation rate, i.e. indicative of vaporized perfluorocarbon nanocarriers. The identified pixels are then displayed over an ultrasound image for anatomical reference, resulting in a photoacoustic-based background-free image (FIG. 8[c]).

### Example 9

### Producing Ultrasound-Based Background-Free Images

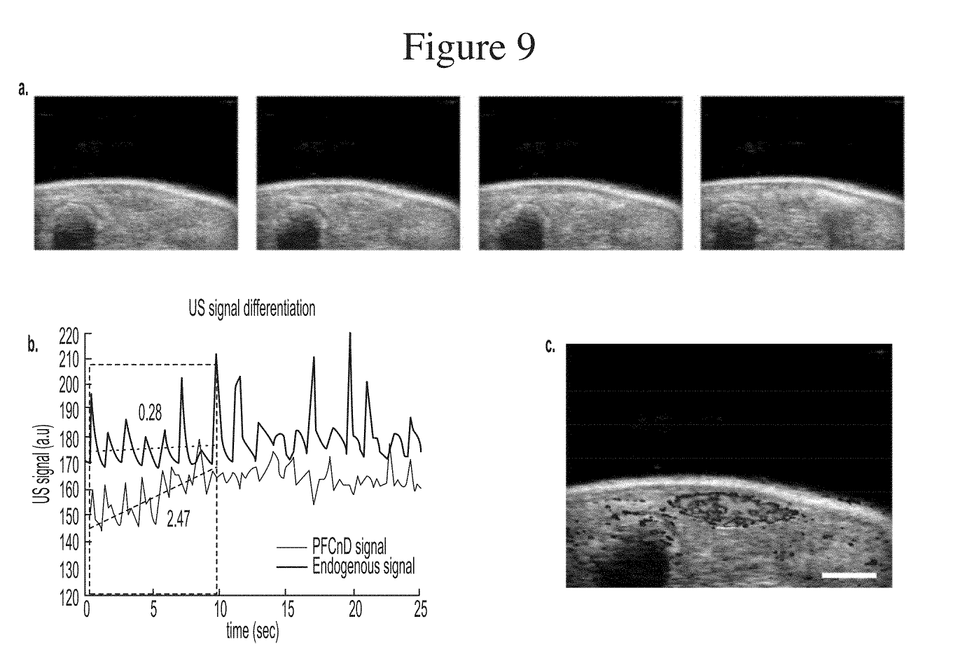

The vaporization-related ultrasound signal of perfluorocarbon nanocarriers mirrors the photoacoustic signal, i.e. increases over time. After the vaporization initiating laser pulse, activated perfluorocarbon nanocarriers begin to transition into microbubbles. However, in order to provide effective ultrasound contrast enhancement, the created microbubbles must first reach a minimum size. This is typically achieved at around 1 depending on the imaging setup and processing available. Consequently, the ultrasound enhancement is less immediate compared to the photoacoustic signal. Similar to photoacoustic signal, for stationary samples the rate of increase is associated with the portion of phase-changed perfluorocarbon nanocarriers at each laser pulse. Faster rates indicate efficient vaporization, whereas slower rates indicate more laser triggers were needed. After some time, the ultrasound signal reaches a maximum and plateaus (FIG. 9).

Background-free ultrasound images are extremely useful for diagnostic applications as they provide a clear special map of the location of the perfluorocarbon nanocarriers. In an analogous method to photoacoustic signal processing, pixels with rapidly increasing ultrasound signal are identified. Frames that capture the ultrasound increase are selected for processing (FIG. 9(b), dashed square), and pixel-wise linear regression is performed.

Pixels which exhibit a slope above a certain threshold are identified as locations where microbubbles were created, i.e. perfluorocarbon nanodroplet phase-change occurred (FIG. 9). The selected pixels are displayed over an ultrasound image for anatomical reference, resulting in an ultrasound-based background-free image (FIG. 9[c]).

Various modifications of the invention, in addition to those described herein, will be apparent to those skilled in the art from the foregoing description. Such modifications are also intended to fall within the scope of the appended claims. Each reference cited in the present application, including all patents, patent applications, and non-patent literature, is incorporated by reference in its entirety.

What is claimed is:

1. A nanocarrier agent comprising:
a perfluorocarbon solution comprising a plurality of nanoparticles dispersed therein; and
a coating material disposed around the exterior surface of the perfluorocarbon solution,
wherein the mean hydrodynamic diameter of the nanocarrier agent is from about 100 nm to about 500 nm.

2. The nanocarrier agent of claim 1, wherein the perfluorocarbon solution comprises a perfluorocarbon selected from the group consisting of: perfluoromethane, perfluoroethane, a perfluoropropane, a perfluorobutane, a perfluoropentane, a perfluorohexane, a perfluoroheptane, perfluoropropene, a perfluorobutene, perfluorobutadiene, perfluorobut-2-yne, perfluorocyclobutane, perfluoromethylcyclobutane, a perfluorodimethylcyclobutane, a perfluorotrimethylcyclobutane, perfluorocyclopentane, perfluoromethycylopentane, a perfluorodimethylcyclopentane, perfluorocyclohexane, perfluoromethylcyclohexane and perfluorocycloheptane, or a combination thereof.

3. The nanocarrier agent of claim 1, wherein the perfluorocarbon is selected from perfluoropentane, perfluorohexane, perfluorobutane, or perfluoroheptane, or a combination thereof.

4. The nanocarrier agent of claim 1, wherein the nanoparticles comprise a metal or metal ion selected from the group consisting of copper, iron, cobalt, gold, silver, and platinum, or a combination thereof.

5. The nanocarrier agent of claim 1, wherein the nanoparticles comprise copper sulfide.

6. The nanocarrier agent of claim 5, wherein the copper sulfide is fluorinated copper sulfide.

7. The nanocarrier agent of claim 1, wherein the nanoparticles have an average diameter of about 0.5 to about 50 nm.

8. The nanocarrier agent of claim 1, wherein the nanoparticles in the perfluorocarbon solution are at a concentration of about 0.05 mg/mL to about 0.3 mg/mL.

9. The nanocarrier agent of claim 1, wherein the coating material is selected from a surfactant, lipid or protein material, or a combination thereof.

10. The nanocarrier agent of claim 1, wherein the coating material is a fluorosurfactant or a polymeric surfactant.

11. The nanocarrier agent of claim 1, wherein the mean hydrodynamic diameter of the nanocarrier agent is about 200 nm.

12. The nanocarrier agent of claim 1, comprising:
a perfluoropentane solution comprising a plurality of fluorinated copper sulfide nanoparticles dispersed therein; and
a fluorinated surfactant coating material disposed around the exterior surface of the perfluoropentane solution,
wherein the mean hydrodynamic diameter of the nanocarrier agent is about 200 nm.

13. A method of preparing the nanocarrier agent of claim 1, comprising:
providing a nanoparticle precursor;
fluorinating the nanoparticle precursor; and
contacting the fluorinated nanoparticle with a perfluorocarbon solution and a coating material to afford a nanocarrier agent.

14. The method of claim 13, wherein the nanoparticle precursor is a citrate-stabilized copper sulfide nanoparticle.

15. A composition comprising an aqueous layer comprising a plurality of nanocarrier agents dispersed therein, the nanocarrier agents comprising
a perfluorocarbon solution comprising a plurality of nanoparticles dispersed therein; and
a coating material disposed around the exterior surface of the perfluorocarbon solution,
the mean hydrodynamic diameter of the nanocarrier agent is about 200 nm, wherein the nanocarrier agents in the aqueous layer are substantially monodisperse.

16. The composition of claim 15, wherein the aqueous layer comprises a saline solution.

17. The composition of claim 15, wherein the aqueous layer is phosphate buffered saline.

18. A method of imaging comprising:
contacting a biological tissue with a nanocarrier agent comprising:
a perfluorocarbon solution comprising a plurality of nanoparticles dispersed therein; and
a coating material disposed around the exterior surface of the perfluorocarbon solution, wherein the mean hydrodynamic diameter of the nanocarrier agent is about 200 nm
applying energy to the tissue, wherein the applying energy results in at least partial vaporization of the nanocarrier agent; and
imaging the biological tissue.

19. The method of claim 18, wherein the imaging comprises application of an imaging technique selected from the group consisting of: photoacoustic imaging, ultrasound imaging, optical imaging, magnetic resonance imaging, computed tomography, thermal imaging, nuclear imaging, magnetomotive imaging enhancement, or a combination thereof.

20. The method of claim 18, wherein the imaging comprises application of photoacoustic imaging or ultrasound imaging.

21. The method of claim 18, wherein the biological tissue comprises human tissue.

22. The method of claim 18, wherein the applying energy comprises irradiating at least a portion of the tissue with a light source or a radio frequency field.

23. The method of claim 18, wherein the nanocarrier agent further comprises a therapeutic agent.

24. The method of claim 23, wherein applying energy to the biological tissue causes a release of the therapeutic agent from the nanocarrier agent.

25. A method of therapy comprising:
contacting a biological tissue with a nanocarrier agent comprising:
a perfluorocarbon solution comprising a plurality of nanoparticles dispersed therein; and
a coating material disposed around the exterior surface of the perfluorocarbon solution, wherein the mean hydrodynamic diameter of the nanocarrier agent is about 200 nm; and
applying energy to the tissue, wherein the applying energy results in at least partial vaporization of the nanocarrier agent.

26. The method of claim 25, wherein the therapy is for treatment of cancer or an inflammatory disease or disorder.

27. The method of claim 26, wherein the inflammatory disease or disorder is dermatitis.

28. The method of claim 26, wherein the therapy is for treatment of cancer.

29. The method of claim 25, wherein the nanocarrier agent comprises a therapeutic agent.

30. The method of claim 29, wherein applying energy to the tissue results in at least partial release of the therapeutic agent from the nanocarrier contrast agent composition.

31. The method of claim 25, wherein applying energy to the tissue comprises irradiating at least a portion of the tissue with a light source or applying a radio frequency field.

32. The method of claim 29, wherein the therapeutic agent is the perfluorocarbon solution or is an agent within the perfluorocarbon solution.

33. The method of claim 29, wherein the therapeutic agent is attached to the exterior surface of the nanocarrier agent.

34. The method of claim 25, wherein the nanocarrier agent further comprises a targeting moiety.

35. The method of claim 34, wherein the targeting moiety is selected from the group consisting of: an antibody, an antibody fragment, a peptide, an aptamer, folate, a ligand, a gene component, and a combination thereof.

36. The method of claim 34, further comprising allowing the nanocarrier agent to accumulate in a region of the biological tissue, wherein the targeting moiety facilitates accumulation of the nanocarrier in the region.

37. The method of claim 18, further comprising processing imaging data by:
collecting photoacoustic signal at different timepoints before and during an irradiation sequence;
identifying pixels of rapidly decreasing photoacoustic signal (slope), during irradiation, below a specified threshold as pixels corresponding to a vaporization of the nanocarrier agent representing nanocarrier-containing pixels;
excluding pixels with a photoacoustic signal slope above a threshold value as endogenous signal; and
displaying identified nanocarrier-containing pixels corresponding to the vaporization of the nanocarrier agent to afford a background-free photoacoustic image.

38. The method of claim 18, further comprising processing imaging data by:
collecting ultrasound signal at different timepoints before and during an irradiation sequence;
identifying pixels of rapidly increasing ultrasound signal (slope), during irradiation, above a specified threshold as pixels corresponding to a vaporization of the nanocarrier agent representing nanocarrier-containing pixels;
excluding pixels with an ultrasound signal slope below a threshold value as endogenous signal; and
displaying identified nanocarrier-containing pixels corresponding to the vaporization of the nanocarrier agent to afford a background-free ultrasound image.

39. The method of claim 25, wherein the applying energy to the tissue containing the nanocarrier or components thereof further provides a photothermal-based therapy.

40. The method of claim 30, wherein the applying energy to the tissue containing the nanocarrier or components thereof further provides a photothermal-based therapy.

41. The nanocarrier of claim 1, wherein nanoparticles are further dispersed within the coating material.
